(19) United States
(12) Patent Application Publication
(10) Pub. No.: US 2023/0330415 A1
(43) Pub. Date: Oct. 19, 2023

(54) SKIN TREATMENT DEVICE

(71) Applicant: Carol Cole Company, Vista, CA (US)

(72) Inventor: Tera Peterson, Vista, CA (US)

(21) Appl. No.: 18/043,279

(22) PCT Filed: Aug. 26, 2021
(86) PCT No.: PCT/US2021/047664

§ 371 Date: Feb. 27, 2023

Related U.S. Application Data
(60) Provisional application No. 63/071,909, filed on Aug. 28, 2020.

(51) Int. Cl. A61N 1/32

(52) U.S. Cl. CPC A61N 1/328

(57) ABSTRACT

A skin treatment device and methods of using thereof are described. The device includes a body and a head. The head is connected to the body. The head includes a skin stimulator, with an outward skin treatment surface configured to contact a user's skin. The device includes a controller configured to provide a skin treatment output signal to the head. In response to the output signal, the skin treatment surface stimulates a user's skin. The skin treatment surface comprises a surface roughness of at least about N3 (e.g., about N3 to about N12).

### INCORPORATION BY REFERENCE TO ANY PRIORITY APPLICATIONS

This application claims priority to and the benefit of Provisional Application No. 63/071,909 filed on Aug. 28, 2020 in the U.S. Patent and Trademark Office, the entire contents of which are incorporated herein by reference.

### BACKGROUND

### Technical Field

The present disclosure relates generally to skin care devices and, more particularly, to a skin toning device (hereinafter to be interchangeably used with a skin treatment device or a skin care device).

### Description of the Related Technology

Stimulators have been used for stimulating muscles, skin, nerves, and hair for the purpose of improving circulation and general rehabilitation. For example, microcurrent or light-therapy device have been used in the cosmetology industry for cellular regeneration (rejuvenation), facial toning and wrinkle reduction. Microcurrent can produce a proliferation of cells by fibroblasts' secretion of proteins such as collagen. Additionally, it works on the nerves and muscle cells to increase tonicity of the fine facial muscles. Research has shown that the results of the microcurrent can be dramatic and long lasting. Microcurrent heals and regenerates tissue; it is a corrective and preventive anti-aging treatment, unlike other relative systems such as muscle stimulators, whose effect is temporary.

As skin ages, cellular functions break down over time. One effect of aging is gradual damage to the DNA and RNA molecules, causing deterioration of the collagen. The end result is that skin is no longer supple and taut as with youthful skin, but rather takes on a dull, sagging, and wrinkled appearance.

By stimulating the skin cells with a skin toning microcurrent device, a user is capable of reducing wrinkles and sagging of the skin, by accelerating collagen, elastin, and connective tissue production, and aid in the regrowth by stimulating mitotic activity and hermetic flow. Thus, any improvements to the microcurrent device that increase these age-reducing features are beneficial.

### SUMMARY

One problem with conventional skin toning devices is that the process of pressing and moving the device on the user's skin can be uncomfortable, due to friction between the user's skin, the head of the device, and the chemical preparations used to activate the device functionality. Described herein are embodiments of skin toning device with heads that have a surface roughness, texture, or dimpling which provides unexpected improvements to comfort and/or skin toning performance.

For purposes of summarizing the invention and the advantages achieved over the prior art, certain objects and advantages of the invention have been described herein above. Of course, it is to be understood that not necessarily all such objects or advantages may be achieved in accordance with any particular embodiment of the invention. Thus, for example, those skilled in the art will recognize that the invention may be embodied or carried out in a manner that achieves or optimizes one advantage or group of advantages as taught herein without necessarily achieving other objects or advantages as may be taught or suggested herein.

All of these embodiments are intended to be within the scope of the invention herein disclosed. These and other embodiments of the present invention will become readily apparent to those skilled in the art from the following detailed description of the preferred embodiments having reference to the attached figures, the invention not being limited to any particular preferred embodiment(s) disclosed.

One aspect is a skin treatment device comprising: a body; a head connected to the body, the head comprising at least one probe with an outward skin treatment surface configured to contact and treat a skin of a user; and a controller configured to generate a microcurrent, the controller further configured to control the at least one probe to conduct the microcurrent along the skin treatment surface and to cause the microcurrent to flow along the skin of the user when the at least one probe contacts the skin of the user, wherein at least a portion of the skin treatment surface comprises a surface roughness of at least about N3.

In the above device, the at least one probe comprises at least a partially spherical distal end. In the above device, the at least one probe further comprises an elongated shaft extending from a base of the head to the at least partially spherical distal end. In the above device, the at least partially spherical distal end comprises an approximately semi-spherical distal end. In the above device, the at least one probe comprises two or more probes. In the above device, the two or more probes comprise only two probes. In the above device, the two or more probes are adjacent to each other and connected with an uninterrupted, curvilinear section of the body.

In the above device, all of the two or more probes extend from a common distal end of the head. In the above device, at least one of the two or more probes is different in size from another one of the two or more probes. In the above device, the head is a detachable head magnetically attached to the body. In the above device, the surface roughness is in the range of about N3 to about N12. In the above device, the controller is configured to generate the microcurrent to have one or more characteristics of a voltage in the range of about 0V to about 20V, a current in the range of about 0 μA to about 1000 μA, or a frequency in the range of about 0.15 Hz to about 450 Hz.

In the above device, the frequency is in the range of about 8.3 Hz-about 8.4 Hz. In the above device, at least the portion of the skin treatment surface is the entirety of the skin treatment surface. In the above device, the outward skin treatment surface is imperforated. In the above device, the outward skin treatment surface is formed of metal. In the above device, the head comprises a base detachably attached to the body, and wherein the at least one probe is fixed to the base when the skin treatment device is in use. The above device further comprises a skin impedance detection sensor configured to detect impedance between the skin treatment surface and the skin of the user when the skin treatment surface contacts the skin of the user.

In the above device, the controller is configured to provide feedback to the user when the detected impedance is less than a threshold impedance. In the above device, the feedback comprises an audio feedback, a visual feedback, or a haptic feedback. In the above device, the feedback recommends the user apply a conductive gel or apply an increased amount of the conductive gel to at least one of the skin treatment surface or the skin of the user to lower the impedance between the skin treatment surface and the skin of the user. In the above device, the controller is configured to generate different intensity levels of the microcurrent that are user selectable, and to increase an intensity level from a currently selected one of the intensity levels in response to receiving a boost treatment command.

Another aspect is a method of using a skin treatment device, comprising: providing a skin treatment device with at least one probe, the at least one probe comprising an outward skin treatment surface, wherein at least a portion of the skin treatment surface comprises a surface roughness of at least about N3; generating a microcurrent; and controlling the at least one probe to conduct the microcurrent along at least the portion of the outward skin treatment surface.

The above method further comprises contacting a skin of a user with the at least one probe to treat the skin of the user while the microcurrent is being conducted along at least the portion of the outward skin treatment surface. The above method further comprises applying a conductive gel to at least one of the skin of the user or the outward skin treatment surface of the at least one probe. In the above method, the conductive gel has a conductive value in the range of about 1.8 milliSiemens/cm to about 6.5 milliSiemens/cm. In the above method, the controlling does not cause at least one of microdermal abrasion or iontophoresis.

The above method further comprises removing a first head from a body of the skin treatment device, and connecting a second head to the body via a magnetic connection. In the above method, the first head comprises at least one first probe including a first outward skin treatment surface having a first surface roughness, and wherein the second head comprises at least one second probe including a second outward skin treatment surface having a second surface roughness different from the first surface roughness.

Another aspect is a skin treatment device comprising: a body; and a head connected to the body, the head comprising at least one probe, the probe comprising an outward skin treatment surface, wherein at least a portion of the skin treatment surface comprises a roughened surface with an arithmetic average roughness of at least about 0.1 μm, wherein the at least one probe is configured to receive a microcurrent and conduct the microcurrent along the roughened surface.

In the above device, the arithmetic average roughness is in the range of about 0.1 μm to about 50 μm.

Another aspect is a head for a skin treatment device comprising: at least one probe, the probe comprising an outward skin treatment surface, wherein at least a portion of the skin treatment surface comprises a roughened surface with an arithmetic average roughness in the range of about 0.1 μm to about 50 μm, wherein the at least one probe is configured to receive a microcurrent and conduct the microcurrent along the roughened surface.

The head is configured to be detachably attached to a body of the skin treatment device.

Another aspect is a skin treatment device comprising: a body; a head connected to the body, the head comprising an outward skin treatment surface configured to contact and treat a skin of a user; a light emitting element disposed on the outward skin treatment surface of the head and configured to emit light for skin treatment; a light emitting element driver configured to enable or disable the light emitting element; a proximity sensor configured to detect proximity or non-proximity in response to whether or not the outward skin treatment surface is adjacent to the skin of the user; and a controller configured to control the light emitting element driver to enable or disable the light emitting element in response to the detected proximity or non-proximity.

The above device further comprises a temperature sensor configured to sense a temperature of the skin treatment device, wherein the controller is further configured to turn off the device in response the sensed temperature being greater than a threshold temperature. In the above device, the controller is configured to enable the proximity sensor and the temperature sensor in response the detected proximity and disable the proximity sensor and the temperature sensor in response the detected non-proximity.

Another aspect is a skin treatment device comprising: a body; a head connected to the body, the head comprising at least one probe with an outward skin treatment surface configured to contact and treat a skin of a user; and a controller configured to generate a microcurrent, the controller further configured to control the at least one probe to conduct the microcurrent along the skin treatment surface and to cause the microcurrent to flow along the skin of the user when the at least one probe contacts the skin of the user, wherein at least a portion of the skin treatment surface comprises at least one of a dimpled surface or a non-smooth textured surface.

In the above device, at least the portion of the skin treatment surface comprises at least one of one or more dimples, one or more ribs, one or more crimps, a scratchy and/or abrasive surface, texture of spheres, or a multi-faceted outer shell.

### BRIEF DESCRIPTION OF THE DRAWINGS

FIG. 1 is a perspective view of an embodiment of a skin toning device.

FIG. 2 is a perspective view of another embodiment of a skin toning device.

FIG. 3 is a perspective view of another embodiment of a skin toning device.

FIG. 4 is a perspective view of another embodiment of a skin toning device.

FIG. 5 is a perspective view of another embodiment of a skin toning device.

FIG. 6 is an example of a surface roughness tester.

FIG. 7 is an example block diagram of another embodiment of a skin toning device.

FIG. 8 is a process flow diagram of an example method of using the skin toning device of FIG. 7.

FIG. 9 is an example block diagram of another embodiment of a skin toning device.

FIGS. 10A and 10B are process flow diagrams of another example method of using the skin toning device of FIG. 9.

FIG. 11 is an example configuration showing different usages of a skin toning device according to some embodiments.

FIG. 12 is an example formula of a conductive gel that can be used for skin toning devices according to some embodiments.

FIGS. 13A-13F show various embodiments of a skin toning device with various stimulator configurations.

### DETAILED DESCRIPTION

Provided herein are various embodiments of skin treatment devices. Some embodiments comprise a non-smooth skin treatment surface on a portion of the treatment head. The non-smooth skin treatment surface may include, for example, a dimpled or otherwise textured surface (e.g., texture of spheres), or other non-smooth surface. The skin treatment devices may include a roughened skin treatment surface. For example, at least a portion of the skin treatment surface comprises a surface roughness of at least about N3, for example, about N3 to about N12. As another example, at least a portion of the skin treatment surface comprises an arithmetic average roughness of at least about 0.1 μm, for example, about 0.1 μm to about 50 μm. Also provided herein are various embodiments of skin treatment devices comprising a light emitting element that can be automatically enabled and disabled depending on whether the skin treatment surface is adjacent to a skin. Embodiments described herein can significantly improve user comfort and/or skin treatment performance.

Although some embodiments will be discussed herein in terms of microcurrent skin toning devices with a certain number of heads or shapes of heads, it will be understood that the invention can also be employed with skin toning devices with other quantities or shapes of heads, or other skin toning technologies.

Various aspects of the novel systems, apparatuses, and methods are described more fully hereinafter with references to the accompanying drawings. Aspects of this disclosure may, however, be embodied in many different forms and should not be construed as limited to any specific structure or function presented throughout this disclosure. Rather, these aspects are provided so that this disclosure will be thorough and complete, and will fully convey the scope of the disclosure to those skilled in the art. Based on the teachings herein, one skilled in the art should appreciate that the scope of the disclosure is intended to cover any aspect of the novel systems, apparatuses, and methods disclosed herein, whether implemented independently of or combined with any other aspect. For example, an apparatus may be implemented or a method may be practiced using any number of the aspects set forth herein. In addition, the scope is intended to encompass apparatuses and/or methods which are practiced using structure and/or functionality in addition to or different than the various aspects specifically set forth herein. It should be understood that any aspect disclosed herein might be embodied by one or more elements of a claim.

Although particular aspects are described herein, many variations and permutations of these aspects fall within the scope of the disclosure. Although some benefits and advantages of the preferred aspects are mentioned, the scope of the disclosure is not intended to be limited to particular benefits, uses, or objectives. The detailed description and drawings are merely illustrative of the disclosure rather than limiting, the scope of the disclosure being defined by the appended claims and equivalents thereof.

Particular implementations of the subject matter described in this disclosure can be implemented to realize one or more of the following potential advantages. The roughness of the skin toning device heads, alone or in combination with the other materials, methods, and/or other features, can provide improved comfort and ease of use, improved iontophoresis, and/or improved anti-aging, anti-wrinkling and other skin toning benefits.

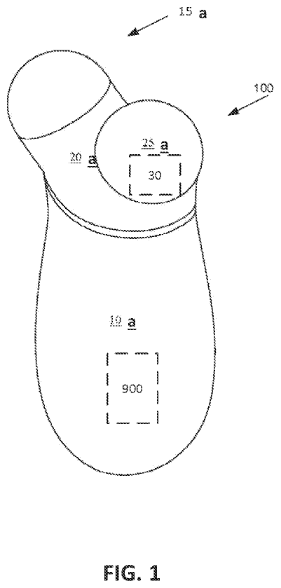

FIG. 1 is a front perspective view of an embodiment of a skin toning device 100. The device 100 includes a body 10a and a head 15a connected to each other. The head 15a can comprise a skin stimulator (e.g., a probe) 25a connected to a base 20a of the head 15a. The stimulator 25a can include an outward skin treatment surface (e.g., skin toning surface) 30 configured to contact a user's skin. FIG. 1 shows only an example position and shape of the skin treatment surface 30, it can be positioned in another location and can have another shape (such as circular, -triangular, rectangular, etc.). Furthermore, Although FIG. 1 shows only one skin treatment surface, there may be two or more skin treatment surfaces separated from each other.

At least a portion of the outward skin treatment surface 30 may include a roughened surface (to be described in greater detail below). In some embodiments, at least the portion of the outward skin treatment surface 30 may be only a portion of the outward skin treatment surface. In other embodiments, at least the portion of the outward skin treatment surface 30 may be the entirety of the outward skin treatment surface 30.

The device 100 can include a controller 900 configured to provide a skin toning output signal (or a skin treatment output signal) to the head 15a. In response to the output signal, the stimulator 25a can stimulate a user's skin, through the contact between the skin toning surface, providing skin toning, wrinkle reduction and other benefits, through the further functionality described herein.

The controller 900 may generate a microcurrent as the skin treatment output signal. The controller 900 may also control the stimulator 25a to conduct the microcurrent along the outward skin treatment surface and may cause the microcurrent to flow along the skin of the user when the outward skin treatment surface 30 of the stimulator 25a contacts the skin of the user, The microcurrent may have one or more characteristics of a voltage in the range of about 0 V-about 20 V, a current in the range of about 0 μA (ampere)-about 1000 μA, or a frequency in the range of about 0.15 Hz-about 450 Hz.

The microcurrent treatment voltage may be in the range of: about 0 V-about 1 V, about 1 V-about 2 V, about 2 V-about 3 V, about 3 V-about 4 V, about 4 V-about 5 V, about 5 V-about 6 V, about 6 V-about 7 V, about 7 V-about 8 V, about 8 V-about 9 V, about 9 V-about 10 V, about 10 V-about 11 V, about 11 V-about 12 V, about 12 V-about 13 V, about 13 V-about 14 V, about 14 V-about 15 V, about 15 V-about 16 V, about 16 V-about 17 V, about 17 V-about 18 V, about 18 V-about 19 V, about 19 V-about 20 V, or any other range between the above described ranges or values. The microcurrent treatment voltage may also be in the range of: about 0 V-about 2 V, about 2 V-about 4 V, about 4 V-about 6 V, about 6 V-about 8 V, about 8 V-about 10 V, about 10 V-about 12 V, about 12 V-about 14 V, about 14 V-about 16 V, about 16 V-about 18 V, about 18 V-about 20 V, or any other range between the above described ranges. The microcurrent treatment voltage may also be in the range of: about 0 V-about 3 V, about 3 V-about 6 V, about 6 V-about 9 V, about 9 V-about 12 V, about 12 V-about 15 V, about 15 V-about 18 V, about 18 V-about 20 V, or any other range between the above described ranges. The microcurrent treatment voltage may also be in the range of: about 0 V-about 5 V, about 5 V-about 10 V, about 10 V-about 15 V, about 15 V-about 20 V, or any other range between the above described ranges. The microcurrent treatment voltage may also be in the range of: about 0 V-about 10 V, about 10 V-about 20 V, or any other range between the above described ranges. Depending on the embodiments, the microcurrent treatment voltage may be greater than about 20 V.

The microcurrent treatment current may be in the range of: about 0 μA-about 100 μA, about 100 μA-about 200 μA, about 200 μA-about 300 μA, about 300 μA-about about 400 μA, about 400 μA-about 500 μA, about 500 μA-about 600 μA, about 600 μA-about 700 μA, about 700 μA-about 800 μA, about 800 μA-about 900 μA, about 900 μA-about 1000 μA, or any other range between the above described ranges or values. The microcurrent treatment current may also be in the range of: about 0 μA-about 150 μA, about 150 μA-about 300 μA, about 300 μA-about 450 μA, about 450 μA-about 600 μA, about 600 μA-about 750 μA, about 750 μA-about 900 μA, about 900 μA about 1000 μA, or any other range between the above described ranges. The microcurrent treatment current may also be in the range of: about 0 μA-about 200 μA, about 200 μA-about 400 μA, about 400 μA-about 600 μA, about 600 μA-about 800 μA, about 800 μA-about 1000 μA, or any other range between the above described ranges. The microcurrent treatment current may be in the range of: about 0 μA-about 300 μA, about 300 μA-about 600 μA, about 600 μA-about 900 μA, about 900 μA-about 1000 μA, or any other range between the above described ranges. Depending on the embodiments, the microcurrent treatment voltage may be greater than about 1000 μA.

The microcurrent treatment frequency may be in the range of: about 0.15 Hz-about 45 Hz, about 45 Hz-about 90 Hz, about 90 Hz-about 135 Hz, about 135 Hz-about 180 Hz, about 180 Hz-about 225 Hz, about 225 Hz-about 270 Hz, about 270 Hz-about 315 Hz, about 315 Hz-about 360 Hz, about 360 Hz-about 405 Hz, about 405 Hz-about 450 Hz, or any other range between the above described ranges or values. The microcurrent treatment frequency may also be in the range of: about 0.15 Hz-about 90 Hz, about 90 Hz-about 180 Hz, about 180 Hz-about 360 Hz, about 360 Hz-about 450 Hz, or any other range between the above described ranges. The microcurrent treatment frequency may also be in the range of: about 0.15 Hz-about 180 Hz, about 180 Hz-about 360 Hz, about 360 Hz-about 450 Hz, or any other range between the above described ranges. The frequency may also be in the range of about 8.3 Hz-about 8.4 Hz, or any other range between the above described range. Depending on the embodiments, the microcurrent treatment frequency may be less than about 0.15 Hz or greater than about 450 Hz.

Furthermore, the microcurrent may have an about 50% duty cycle, and 10 positive pulses followed by about 10 negative pulses. The microcurrent may also have an about 50% duty cycle, and any one of about 10 positive pulses-about 50 positive pulses followed by any one of about 10 negative pulses-about 50 negative pulses. Depending on the embodiment, the microcurrent may have a duty cycle more than or less than an about 50% duty, cycle (e.g., about 40% or about 60% duty cycle, etc.). Depending on the embodiment, each of the microcurrent positive and negative pulses may be less than about 10 pulses or more than about 50 pulses.

As described below, a strength and duration of the microcurrent may be user-adjustable. The controller 900 may be located in the body 10a as shown FIG. 1. However, the controller 900 may be located in the head 15a, or both in the body 10a and the head 15a.

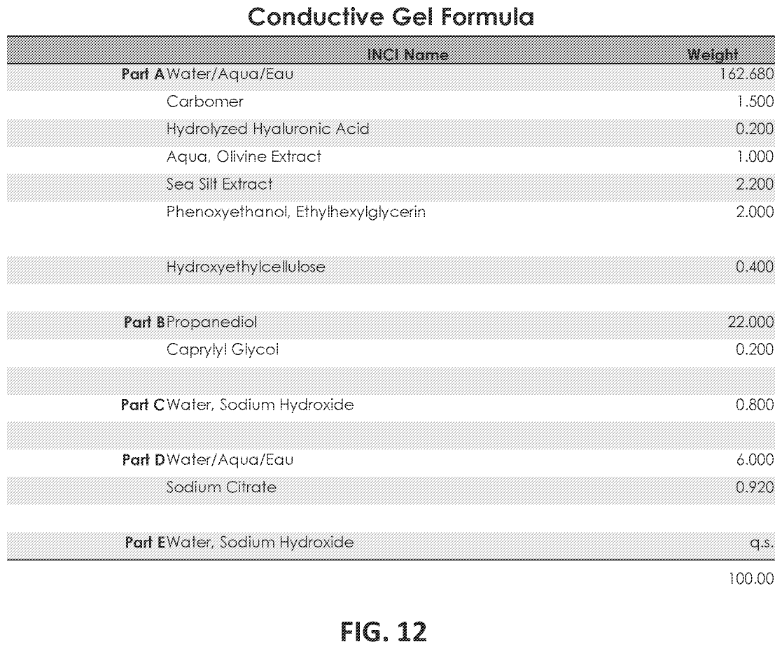

Each of the above microcurrent treatment voltage ranges may be combined with one or more of: any one of the microcurrent treatment current ranges, any one of the microcurrent treatment frequency ranges, any one of the microcurrent duty cycle ranges, or any one of conductive value ranges of a conductive gel (to be described with respect to FIG. 12). Each of the above microcurrent treatment current ranges may be combined with one or more of: any one of the microcurrent treatment voltage ranges, any one of the microcurrent treatment frequency ranges, any one of the microcurrent duty cycle ranges, or any one of conductive value ranges. Each of the above microcurrent treatment frequency ranges may be combined with one or more of: any one of the microcurrent treatment voltage ranges, any one of the microcurrent treatment current ranges, any one of the microcurrent duty cycle ranges, or any one of conductive value ranges. Each of the above microcurrent duty cycle ranges may be combined with one or more of: any one of the microcurrent treatment voltage ranges, any one of the microcurrent treatment current ranges, any one of the microcurrent treatment frequency ranges, or any one of conductive value ranges. Certain combinations of two or more of the microcurrent treatment voltage ranges, the microcurrent treatment current ranges, the microcurrent treatment frequency ranges, or the microcurrent duty cycle ranges, or conductive value ranges may provide certain advantages including, but not limited to, enhanced user comfort and/or better skin treatment performance. One or more of the microcurrent treatment voltage ranges, the microcurrent treatment current ranges, the microcurrent treatment frequency ranges, or the microcurrent duty cycle ranges, or conductive value ranges may also be combined with a certain ranges of surface roughness or a certain surface roughness, to provide certain advantages described above, or other advantages.

The body 10a and the head 15a can have various shapes. In some embodiments, the body 10a and the head 15a are sized and shaped such that the device 100 is a hand held device, By “hand held” it is meant that the entire device 100 may be easily grasped and manipulated for its intended skin toning purpose in one average-sized adult human hand. The body 10a and the head 15a can be integrally formed. The body 10a and the head 15a can be detachably coupled to each other. The detachable coupling may be, for example, with magnetic attachments. However, other detachable coupling mechanism such as screw coupling, push-to-lock, or twist to lock coupling may also be used. Such detachable coupling can allow the body 10a to be implemented with various types of interchangeable heads, to provide various skin toning functionality as described elsewhere herein.

The device 100 may include two or more interchangeable heads. For the purpose of convenience of description, it is assumed that there are two interchangeable heads, although there may be more than two interchangeable heads. In some embodiment, when a first head is attached to the device 100, a user may remove the first head from the body 10a and connect a second head to the body 10a. The first head may include at least one first probe or stimulator including a first outward skin treatment surface having a first surface roughness such as about 3N or about 5N, or other values or ranges described herein. The second head may include at least one second probe including a second outward skin treatment surface having a second different surface roughness such as about 10N or about 12N, or other values or ranges described herein.

The first head may include a first number of first probes and the second head may include a second number of second probes. In some embodiments, the first number and the second number may be the same. In these embodiments, each of the first probes may have a surface roughness that is different from that of each of the second probes.

In other embodiments, the first number and the second number may be different. In these embodiments, the first and second probes may have the same degree of surface roughness or different degrees of surface roughness relative to each other.

In some embodiments, although the head 15a is detachable, the head 15a is fixed to the body 10a, when the device 100 is in a “use” position. The probe 25a may be fixed relative to the body 10a, or to the remainder of the head 15a, to avoid motion relative to the body 10a , when the device 100 is in a “use” position.

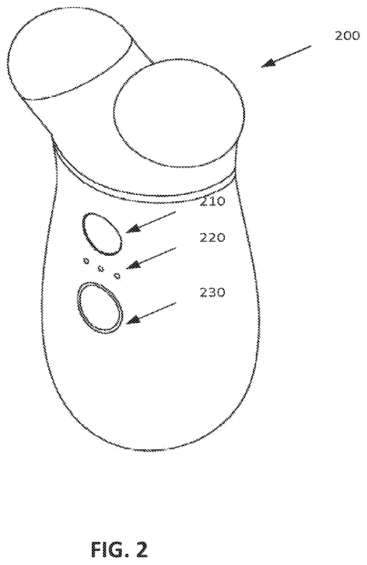

The probe 25a may be solid or non-hollow, or may be a hollow shell. The outward skin treatment surface 30 of the probe 25a may be imperforated. In some embodiments, as shown in FIGS. 1 and 2, when there are two or more probes, all of them may extend from a common distal end of the base 20a of the head 15a.

The body 10a can include a housing to contain the controller 900, a battery, and other electrical, mechanical, electro-mechanical, or other components to provide the functional features herein. In some embodiments, the body 10a and/or the head 15a can be waterproof, for example to meet a minimum of IP56 requirements, or even IP57 or better.

The controller 900 can be configured to provide the functionality of the methods described herein, and additional functionality. Depending on the embodiment, the controller 900 may include a processor and a memory storing instructions configured to cause the processor to execute the methods described herein. For example, the controller 900 can be configured to communicate (e.g., electronically) with the other electrical, mechanical or electro-mechanical apparatus, and/or other skin toning device equipment components described herein, or other systems or components, to provide functionality thereto. An example with more details of circuitry and other features and components that can be implemented within the context of the controller 900 and the skin toning devices herein is described in International PCT Pub. No. WO2006/116728, the entirety of which is incorporated by reference and provided as Appendix A.

In some embodiments, the controller 900 is configured to allow the frequency of the microcurrent being delivered from the device to be adjustable, for example, within a range of between approximately about 0.15 hertz (Hz)-about 450 Hz, to provide further treatment options for a device intended to operate within that range. In some embodiments, one or more of the microcurrent treatment voltage, current, frequency may be adjustable by a user, for example, via an adjustment switch or a mobile or smartphone app (to be described later). In other embodiments, the frequency of the microcurrent may be automatically adjustable by the controller 900. The automatic adjustment may be based on one or more of pre-programmed operation modes. The controller and/or other circuitry can be configured to connect (e.g., remotely) the device 100 to another device, such as a personal computer or mobile phone. For example, the device 100 can be Bluetooth-enabled, and/or can interface with another user interface, such as a user interface on a mobile device, using a mobile app. The device 100 can be configured to have inductive charging to its battery.

The stimulator 25a can comprise various shapes and configurations. The stimulator 25a herein can be configured to deliver microcurrent or other stimulation to a user's skin to provide toning or skin treatment benefits. In some embodiments, the stimulator 25a can comprise at least one probe extending outwardly from the base 20a of the head 15a, as shown. The probe can deliver microcurrent to a user's skin. In some embodiments, the stimulator 25a can comprise a probe with a spherical or at least a partially spherical (e.g., a spherical dome) distal end. For example, the probe can comprise an approximately semi-spherical distal end, as shown.

Various quantities of probes can be implemented, such as two or more probes (e.g., 2, 3, 4, 5 or more probes), or only two probes, as shown. The stimulator 25a can comprise various materials, such as plastic or metal. In some embodiments, a base metal of the stimulator can comprise Zn, Al, Cu, Mg, Fe, Pb, Cd and/or Sn. In some embodiments, a base metal of the stimulator is a base alloy and can comprise a Zn alloy, Al ahoy, Cu alloy (e.g. brass alloy), Mg alloy, and/or Fe alloy. For example, the base metal or alloy of the stimulator can comprise Zamak 3 (ASTM AG40A, ZnAl4) or Zamak ZA 8, HBI59-1 alloy, or H59-1. The base metal or alloy of the stimulator can be coated. In some embodiments, the base metal or alloy of the stimulator is coated with a coating metal comprising Co, Cr, Au, Ag, Cu, and/or Zn. In some embodiments, the base metal or alloy of the stimulator is coated. with a coating metal alloy comprising a Co alloy, Cr alloy, Au alloy, Ag alloy, and/or Cu ahoy. For example, the base metal or alloy of the stimulator may be coated with chrome, or gold (e.g. yellow gold). In some embodiments, coating may be performed though electrodeposition of the base metal. For example, chrome may be electrodeposited utilizing ISO 6158, ZN//CR14-18HR, rose gold may be electrodeposited utilizing Endura Glo 520 Electrolyte, and gold (e.g., yellow) may be electrodeposited utilizing Rona Flash 1N electrolyte.

FIG. 2 is an embodiment of a skin toning device 200 that is substantially similar to that shown in FIG. 1, with additional components. FIG. 2 shows only an example arrangement of the elements 210-230, and other arrangement of the elements 210-230 may also be possible. In some embodiments, the button 210 may be a boost only button whereas the button 230 may be a power/level change button. In other embodiments, the button 210 may be a power/level change button whereas the button 230 may be a boost only button. Although the elements 210-230 are vertically arranged in FIG. 2, the elements 210-230 may be horizontally arranged, or in other orientations. Furthermore, one or two of the elements 210-230 may be located on another side of the body of the device 200, or on the same or different side, relative to each other.

Here, the device 200 can include a power button 230 configured to turn the device ON and OFF. The power button 230 can also be configured to switch between multiple levels (e.g., 3 levels) of intensity when the device 200 is ON. The device 200 can include indicators 220 configured to signify multiple different user experiences depending on the state of the device 200. The indicators 220 may comprise one or more light indicators such as LED indicators. For example, when the device 200 is turned on, the light indicators 220 can indicate the level of treatment a user is utilizing (e.g., low—first LED lit only, Medium—first and second LED lit only, High—all three LED lit). When the device 200 is off if the user holds the power button 230 down for, for example, about 1 second the indicators 220 can indicate battery state (e.g., low battery—first LED lit only, Medium—first and second LED lit only, High—all three LED lit). When the user holds the power button 230 down for, for example, about 5 seconds the device 200 can enter pairing mode for Bluetooth or other remote capability, and the lights can fade on and off from side to side to show the device 200 is trying to connect to another device. Once paired, the LEDs 220 can simultaneously blink multiple times (e.g., 3 times). When the device 200 is connected to an app (e.g., an external app, such as a mobile app), for example, to synchronize a treatment, the device 200 can alternate blinking the outer 2 LEDs with the inner LED to indicate the program is switching over and then blink simultaneously, for example, 3 times to indicate the program is ready. Lastly when the device 200 is connected to its charging cradle, the LED 220 can light up left to right to show the device 200 is charging, and can become full (e.g., all three LEDs lit) once the battery is fully charged. If the power button 230 is held down, for example, for 10 seconds while the device 200 is connected to the charging cradle, the device may perform a factory reset and the outer LED's will blink, for example, 3 times to show success.

The device 200 can include a boost switch 210 configured to turn on the boost treatment when the device 200 is in the ON state. The switch 210 may not be activated if the device 200 is off. When the switch 210 is activated, the microcurrent output may, increase by a certain percentage, for example, about 25-about 50%, depending on user selections. The above boost percentages are merely examples, and the present disclosure is not limited thereto. For example, depending on the embodiment, the boost percentage may be less than about 25% or greater than about 50%. When the boost switch 210 is turned on, feedback (e.g., audio, visual, haptic, etc.) may be provided to let the user know the boost treatment is ON. The switch button 210 is pressed again to turn the boost OFF.

The boost treatment microcurrent can be higher than a level of the microcurrent previously set by the user. For example, when the device 200 is in a first intensity level (e.g., 72 μA) before the boost switch 210 is on, the boost microcurrent can be, for example, 108 μA. The boost microcurrent at the first intensity level may be lower or higher than a second intensity level of the microcurrent. As another example, when the device 200 is in a second intensity level (e.g., 101 μA) before the boost switch 210 is on, the boost microcurrent can be, for example, 150 μA. The boost microcurrent at the second intensity level may be lower or higher than a third intensity level of the microcurrent. As another example, when the device 200 is in a third intensity level (e.g., 170 μA) before the boost switch is On, the boost microcurrent can be, for example, 255 μA. The above microcurrent values of the first to third intensity levels are merely examples, and other microcurrent values are also possible. Furthermore, the above boost microcurrent values are also merely examples, and other boost microcurrent values are also possible. In some embodiments, two or more boost microcurrent intensity levels may be possible. For example, pushing the boost button once may provide a first boost microcurrent level, and pushing the boost button twice or more may provide a second or higher boost microcurrent level. As another example, pressing and holding the boost button for a first period of time may provide a first boost microcurrent level, and pressing and holding the boost button for a second or longer period of time may provide a second or higher boost microcurrent level.

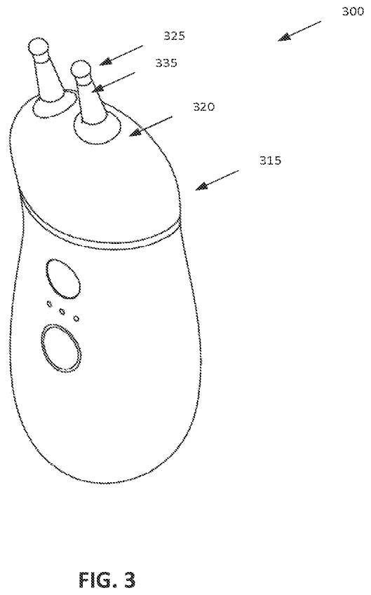

FIG. 3 is an embodiment of a skin toning device 300 that is substantially similar to that shown in FIG. 1 or 2, with the following differences. Here, the device 300 includes a stimulator 325 comprising a probe with at least a partially spherical distal end and an elongated shaft 335 extending from a base 320 of the head 315 to the at least partially spherical distal end. The device 300 can be used for more focused skin toning, such as eye wrinkles. Although not shown in FIG. 3, the device 300 may include at least one stimulator 325 and at least one another larger stimulator such as the stimulator 25a shown in FIG. 1. In these embodiments, the device 300 may be used for skin treatment for a relatively large body portion (e.g., face, arm neck, leg, etc.) with the stimulator 25a and for a relatively small body portion (e.g., eye or nose areas) with the stimulator 325. The stimulator 25a and the stimulator 325 may have different degrees of surface roughness or the same degree of surface roughness. According to these embodiments, a user can perform dual purpose skin treatment for smaller and larger body areas without the need of replacing heads.

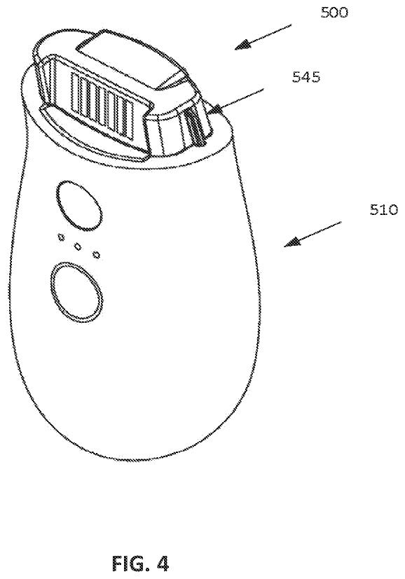

FIG. 4 is an embodiment of a skin toning device 500 with a body 510 without a head attached thereto. The body 510 of the device 500 can include an electrical connector 545 to electrically interface with a corresponding skin toning device head. The body 510 and/or electrical connector 545 to facilitate removal of the head can be implemented with any of the skin toning devices shown in FIGS. 1-3. For example, the head from FIG. 1 can be removed from the body shown in FIGS. 1 and 4, and replaced with the head shown in FIG. 3, and so forth. Thus, the body 510 and/or connector 545 can be implemented within any of the heads and devices shown in FIGS. 1-4. Different heads can be attached to and detached from the body 510 of the device 500. Different coupling/decoupling mechanism can be used as described above. Again, the head may be fixed to the body 510 and thus may not move with respect to the body 510 while the device 500 is in use.

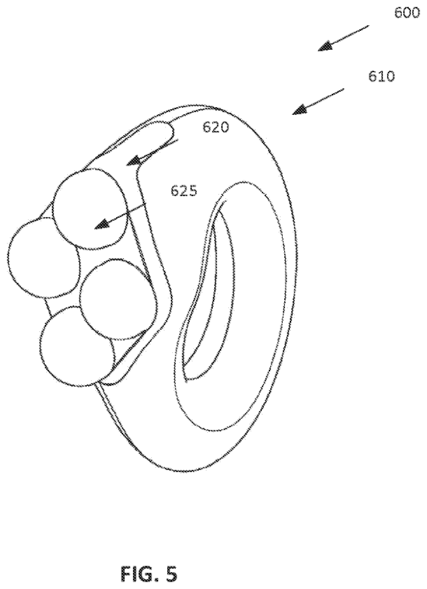

FIG. 5 is an embodiment of a skin toning device 600, which can be similar to and can include many of the features of FIGS. 1-4. Here, the skin toning device 600 can include a body 610 with an opening extending therethrough, to provide a handle. Device 600 can include a head 620 with a plurality of stimulators, for example, four stimulators 625. The stimulators can comprise probes, as described in other embodiments. The device 600 can be larger than the embodiments shown in FIGS. 1-4, for example, for use on other areas of skin than a user's face. In some embodiments, skin treatment surfaces of all of the stimulators 625 may have the same degree of surface roughness. In other embodiments, at least one of the stimulators 625 may include a roughened skin treatment surface different from that of the remaining stimulators.

The following surface features can be implemented within any of the embodiments described herein, or other skin toning devices that include a skin stimulator with a corresponding skin toning surface:

The skin toning surfaces describe herein are defined as the outward-facing surface of the stimulators which, during use of the skin toning device, can contact a user's skin and provide the skin toning benefits thereto, in response to the skin toning output signal to the head from the controller. The skin toning surface can include substantially flat portions, substantially curvilinear portions, or both. The skin toning surface can comprise a surface with a surface roughness that is above certain thresholds and within certain ranges, which, when used in combination with various chemical applicator preparations, are discovered to unexpectedly provide a smoother, more comfortable application to the user's skin, and/or increased skin toning benefits. The comfort and/or improved skin toning benefits provided by such a roughened surface was counterintuitive to the previous understanding by those of ordinary skill in the art for conventional stimulators, in which a smoother surface was perceived to provide high comfort levels and skin toning benefits. Smoother surfaces were also implemented to reduce wear and increase device life.

The roughness values of the surface can be defined as a surface roughness value N, under the ISO 4287:1997 standard, based on the mean line (“M”) system. As such, the surface 30 can comprise a surface roughness N of about N3 to about N12, or about N3 to about N5, or about N6 to about N9, for example, about or at least about, N3, or about or at least about N4, or about or at least about N5, or about or at least about N6, or about or at least about N7, or about or at least about N8, or about or at least about N9 or about or at least about N10, or about or at least about N11, or about or at least about N12, or any other range between the above described ranges or values. The surface roughness may be greater than about N12 depending on embodiments, as long as it is at least about N3.

In some embodiments, different degrees of surface roughness may be used with different characteristics of the microcurrent. For example, lower ranges or values of the voltage (about 0 V-about 20 V), lower ranges or values of the current (about 0 μA-about 1000 μA), lower ranges or values of the frequency (about 0.15 Hz-about 450 Hz), lower ranges or values of the microcurrent pulse (about 10-about 50 positive or negative pulses), and/or lower ranges or values of the duty cycles (about 40%-about 60%) may be used with a lower degree of surface roughness (e.g., about 3N-about 8N, or any other surface roughness therebetween), or a higher degree of surface roughness (e.g., about 8N-about 12N, or any other surface roughness therebetween). As another example, higher ranges or values of the voltage (about 0 V-about 20 V), higher ranges or values of the current (about 0 μA-about 1000 μA), higher ranges or values of the frequency (about 0.15 Hz-about 450 Hz), higher ranges or values of the microcurrent pulse (about 10-about 50 positive or negative pulses), and/or higher ranges or values of the duty cycles (about 40%-about 60%) may be used with a higher degree of surface roughness (e.g., about 8N-about 12N, or any other surface roughness therebetween), or a lower degree of surface roughness (e.g., about 3N-about 8N, or any other surface roughness therebetween). At least some combinations of the above ranges may provide some benefit such as improved skin toning performance and/or enhanced user comport.

Under the same ISO 4287:1997 standard, the skin treatment surface 30 can comprise an arithmetic average roughness Rₐ of about 0.1 μm to about 50 μm, for example, about 0.15 μm to about 0.7 μm, about 0.8 μm to about 4.4 μm, about or at least about 0.3 μm, about or at least about 0.6 μm, about or at least about 0.7 μm, about or at least about 0.8 μm, about or at least about 0.9 μm, about or at least about 1.0 μm, about or at least about 1.25 μm, about or at least about 1.5 μm, about or at least about 1.75 μm, about or at least about 2.0 μm, about or at least about 2.25 μm, about or at least about 2.5 μm, about or at least about 2.75 μm, about or at least about 3.0 μm, about or at least about 3.2.5 μm, about or at least about 3.5 μm, about or at least about 3.75 μm, about or at least about 4.0 μm, about or at least about 4.25 μm, about or at least about 4.5 μm, about or at least about 10 μm, about or at least about 15 μm, about or at least about 20 μm, about or at least about 25 μm, about or at least about 30 μm, about or at least about 35 μm, about or at least about 40 μm, about or at least about 45 μm, about or at least about 50 μm, or any other range between the above described ranges or values. Depending on the embodiment, the arithmetic average roughness may be less than about 0.1 μm or greater than about 50 μm.

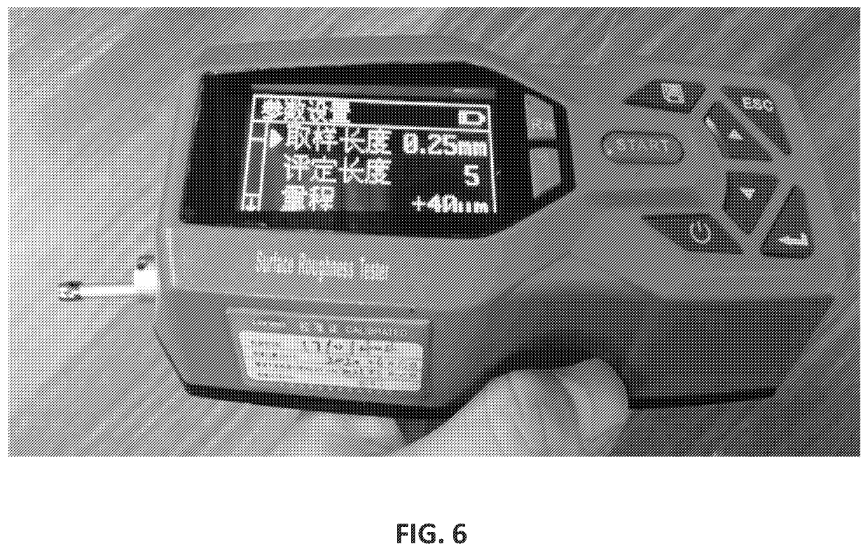

The roughness of the skin toning surfaces of the skin stimulators herein can be measured with a roughness tester, such as shown, for example, in FIG. 6. The roughness of the surfaces can be achieved through various material processing techniques, such as, for example, a bead blast machine. In an example, a bead blast with φ0.6 mm/SS shot achieved a surface roughness Rₐ between about 3.3 μm and about 4.1 μm, and in another example, a bead blast with φ0.15 mm/SS shot resulted in a roughness Rₐ of between about 1.1 μm and about 1.3 μm.

The skin toning devices herein can be configured to be used with, or can be provided as a kit with various types of chemical preparations. A conductive gel may be implemented, for example, for those embodiments that provide microcurrent to the skin. Other preparations, such as cosmetic preparations, may provide additional benefits. In some embodiments, the skin toning device can be used or provided as a kit with a cosmetic preparation and a conductive gel, as separate preparations, or a compound/mixture that includes both.

The skin toning devices herein can be used through the following steps:
Provide a skin toning device with a skin stimulator. The skin stimulator has a surface roughness of at least about N3.
Contact a user's skin with the skin stimulator.
Tone user's skin with the skin stimulator. To tone the user's skin, a microcurrent can be applied to user's skin with the stimulator. Alternatively, light can be applied to the user's skin.
In some embodiments, remove a first head from a body of the skin toning device, and connect a second head to the body via a magnetic connection.

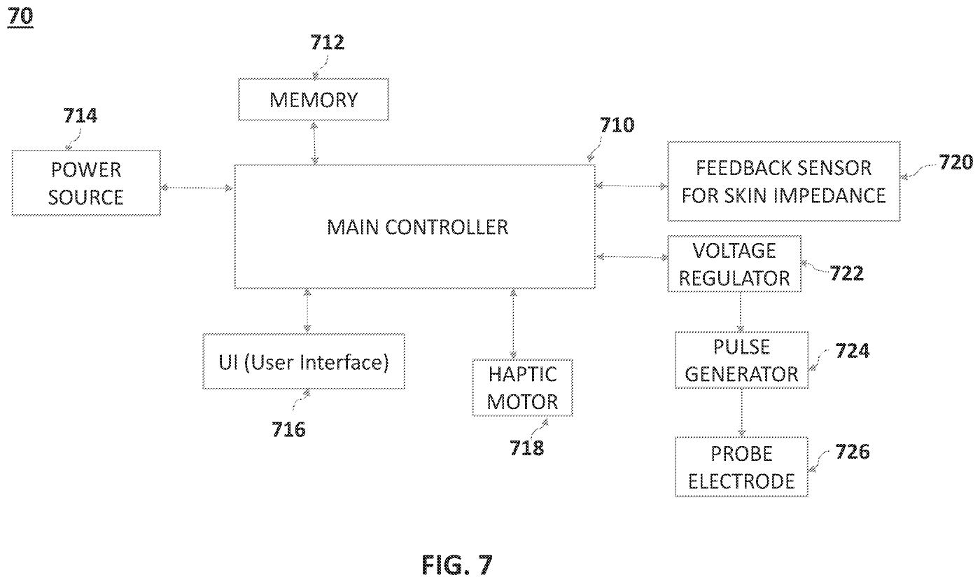

FIG. 7 is a block diagram of another embodiment of a skin toning device 70. Device 70 can be similar to and can include many of the features of the other skin toning devices herein, and its features and methods of operations can be implemented with the other skin toning devices herein. The skin toning device 70 may include a main controller 710, a memory 712, a power source 714, a user interface (UI) 716, a haptic motor 718, a feedback sensor for skin impedance 720, a voltage regulator 722, a pulse generator 724, and a probe electrode 726. The elements 710-726 may directly or indirectly communicate data with each other. The skin toning device 70 shown in FIG. 7 is merely an example skin toning device, and certain elements may be modified or removed, two or more elements combined into a single element, and/or other elements may be added.

The main controller 710 may control an overall operation of the skin toning device 70. The main controller 710 can be configured to provide the functionality of the methods described herein, and additional functionality. The main controller 710 may execute instructions stored in the memory 712 to perform various operations of the skin toning device 70 described herein. The main controller 710 may be located in the body, the head, or both in the body and the head of the skin toning device 70.

The memory 712 may communicate data with the main controller 710. The memory may store the executable instructions relating to the various operations of the skin toning device 70 so that the main controller 710 can execute the instructions to perform the various operations of the skin toning device 70.

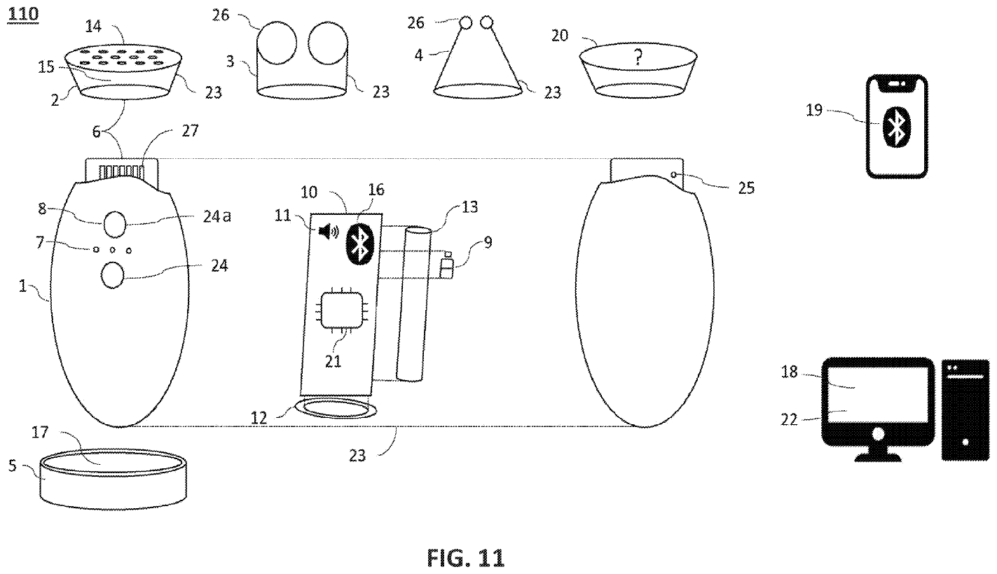

The power source 714 may provide power to one or more of the remaining elements of the skin toning device 70. The power source 714 may include, but is not limited to, a battery, a rechargeable battery, or a power outlet. The rechargeable battery may be charged wirelessly or by wire. The power source 714 may include a voltage adapter (e.g., 9V, 12V, or 15V, etc.) that converts voltage from a power outlet into a lower voltage that is suitable to operate the skin toning device 70. The power source 714 may include a wired or wireless charging base such as a charging cradle (to be described in more detail with respect to FIG. 11).

The UI 716 may provide various types of user interface between a patient or user and the skin toning device 70. The UI 716 may include various software and/or hardware components interfacing between a patient and the skin toning device 70. The various software and/or hardware components may include, but are not limited to, a tactile power switch (e.g., 230 in FIG. 2), a tactile boost switch (e.g., 210 in FIG. 2), light indicators (e.g., 220 in FIG. 2), a Bluetooth module (e.g., 16 in FIG. 11), a wireless charging status indicator, a battery power status indicator, audio, or a motor such as a haptic/tactile motor.

The haptic motor 718 may provide a haptic feedback, such as force or vibration, to the patient. The main controller 710 may control the haptic motor 718 to provide the haptic feedback in various situations that may be desirable to notify the patient about a haptic feedback. For example, the haptic motor 718 may provide a haptic feedback when a boost treatment is enabled so that the patient can recognize that the skin toning device 70 is in a boost mode. The haptic motor 718 may also provide a haptic feedback when the skin toning device 70 is turned on or turned off, has a low level of a battery, or switched to another microcurrent intensity level. The haptic motor 718 may also provide a haptic feedback when the skin toning device 70 is moved from one area e.g., forehead, eyelid, etc.) of the patient's skin to another area (e.g., cheek, nose, etc.) of the patient's skin. In these embodiments, the skin toning device 70 may include a motion sensor that can recognize a movement of the skin toning device 70 when the skin toning device 70 moves for more than a predetermined distance. The above are merely example feedback conditions where the main controller 710 controls the haptic motor 718 to provide a haptic feedback, and other feedback conditions are also possible. In some embodiments, the memory 712 stores a list of haptic feedback conditions, and the main controller 710 may control the haptic motor 718 to provide a corresponding haptic feedback when one or more of the stored haptic feedback conditions occur. In some embodiments, the haptic motor may also be used as a massager. In other embodiments, the skin toning device 70 may include an additional vibration motor that is used for massaging the skin of the user. The UI 716 may also provide feedback (audio, visual, haptic) when one or more feedback conditions described above occur. In some embodiments, the haptic motor is of sufficient power to provide the aforementioned feedback to a user, but is below a power threshold for the device to be used as a massager. In some embodiments, the skin toning device includes only a single, haptic motor (i.e., without a second motor). In some embodiments, the skin toning device includes only a single, haptic motor (i.e., without a second motor), wherein the single haptic motor is only configured to provide the above haptic feedback as to the above functionality of the device (i.e., without massage capability).

Regardless of whether the skin toning device 70 includes a motor such as the haptic motor 718, the skin toning device 70 may be configured to provide feedback to alert the user to move the device 70 to a next treatment area (like an electric toothbrush) when the friction between the skin treatment surface and the skin of the user is above a threshold that would cause microdermal abrasion to the skin.

The feedback sensor for skin impedance 720 may detect a level of skin impedance while the skin toning device 70 contacts a patient's skin. The feedback sensor 720 may include a resistive sensor, a capacitive sensor, or other skin impedance sensor that can detect a level of impedance between the patient's skin and the treatment surface of the skin toning device 70. Low impedance may indicate that there is a good contact between the patient's skin and the treatment surface. Furthermore, high impedance may indicate that there is a poor contact between the patient's skin and the treatment surface. Applying a conductive gel may improve skin impedance to provide a better contact between the patient's skin and the treatment surface. In some embodiments, when high impedance is detected, the main controller 710 may control the UI 716 and/or the haptic motor 718 to provide feedback such as audio, visual (e.g., using one or more of indicator LEDs), or haptic feedback so that the patient may be advised to apply more conductive gel. In other embodiments, when low impedance is detected, the main controller 710 may control the UI 716 and/or the haptic motor 718 to provide feedback indicating that there is a good or improved contact between the patient's skin and the treatment surface.

The voltage regulator 722 may regulate one or more of the microcurrent treatment voltage, the microcurrent treatment current, the microcurrent treatment frequency, or the microcurrent pulse or duty cycle, as described above. The main controller 710 may control the operation of the voltage regulator 722.

The pulse generator 724 may generate a microcurrent pulse under the control of the voltage generator 722. The probe electrode 726 may receive the microcurrent pulse from the pulse generator and flow the microcurrent along the skin treatment surface of the skin toning device 70 when the skin treatment surface contacts the patient's skin. The probe electrode 726 may be disposed inside or on the skin treatment surface of the head. The probe electrode 726 may include a single electrode or a plurality of electrodes along which the microcurrent flows, depending on the embodiment.

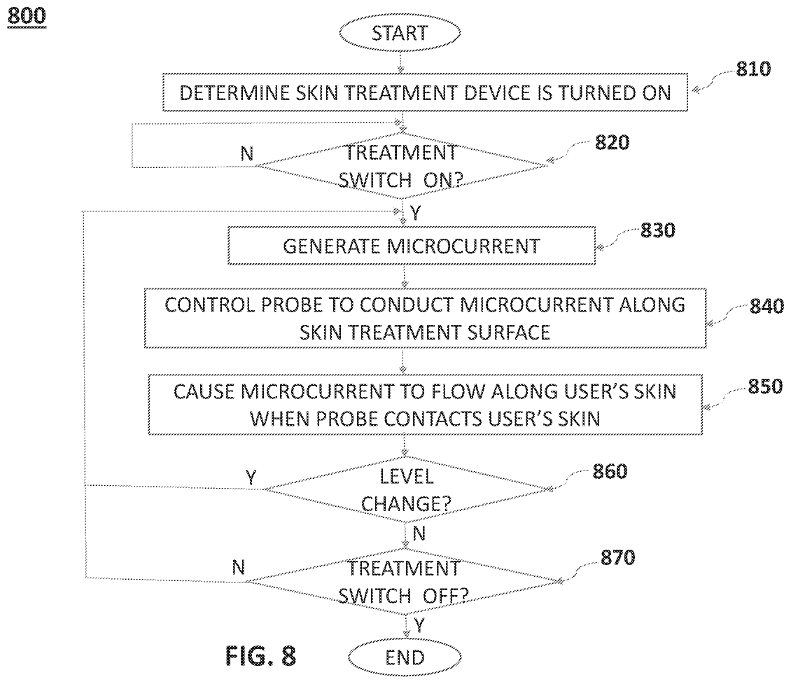

FIG. 8 is a process flow diagram 800 of an example method of operating the skin toning device 70 of FIG. 7. The process flow diagram 800 may be performed by, for example, one or more components (e.g., main controller 710) of the skin toning device 70 of FIG. 7. FIG. 8 is merely an example process flow diagram for a method of operating the skin toning device 70, and certain states elements may be removed, other states added, two or more states combined or one state can be separated into multiple states depending on the specification and requirements. For the purpose of the convenience, the description will be made based on the main controller 710 shown in FIG. 7 performing the process flow diagram 800 of FIG. 8.

In state 810, the main controller 710 may determine that the skin toning device 70 is turned on. For example, when the power button is pressed, the main controller 710 may determine that the skin toning device is turned on. In state 820, the main controller 710 may determine whether the treatment switch is on. Once the skin toning device is turned on, the power button 210 can also function as a treatment switch. For example, when the power button 210 is pressed after the skin toning device is turned on, the skin toning device can be switched between multiple intensity levels (e.g., 3 levels). When it is determined in state 820 that the treatment switch is not turned on, state 820 may repeat until the treatment switch is turned on.

When it is determined in state 820 that the treatment switch is turned on, the main controller 710 may generate microcurrent (state 830). The microcurrent may initially have a first level that may correspond to a first intensity level that can be set as a default intensity by the power button/treatment switch. For example, when the power button is pressed once after the skin toning device 70 is turned on, the intensity level of the microcurrent may be set as a first intensity level. As another example, when the power button is pressed and held for a predetermined period of time (e.g., 2 seconds) after the skin toning device 70 is turned on, the intensity level of the microcurrent may be set as a first intensity level.

In state 840, the main controller 710 may control the probe/stimulator to conduct microcurrent along the skin treatment surface 30 (see FIG. 1). For example, the main controller 710 may deliver the generated microcurrent to at least the skin treatment surface 30 of the probe/stimulator (e.g., 25a in FIG. 1). In state 850, the main controller 710 may cause the delivered microcurrent to flow along a user's skin when the skin treatment surface 30 of the probe/stimulator 25a contacts the user's skin.

In state 860, the main controller 710 may determine whether the intensity level of the microcurrent has been changed from the first intensity level to another level. For example, when the power/level switch button is pressed again while the skin toning device 70 is operating at the first intensity level, the intensity level of the microcurrent may change to a second intensity level different from the first intensity level. As another example, when the power button 210 is pressed and held for a predetermined period of time (e.g., 2 or 3 seconds) while the skin toning device is operating at the first intensity level, the intensity level of the microcurrent may change to a second intensity level different from the first intensity level. The second intensity level may be higher or lower than the first intensity level. Furthermore, as described above, the level change may be implemented by a boost treatment process. That is, when a boost button (e.g., 210 in FIG. 2) is pressed, a microcurrent intensity level is changed to a higher level. When it is determined in state 860 that the treatment level changes to another level, the main controller 710 may generate microcurrent corresponding to the changed intensity level (e.g., a second or third intensity, level, or a boost microcurrent level). Thereafter, the main controller 710 may repeat states 830-860.

When it is determined in state 860 that the treatment level has not changed to another level, the main controller 710 may determine whether the treatment switch has been turned off (state 870). For example, when the power button/treatment switch is pressed again while the skin toning device 70 is operating at a maximum intensity level (e.g., third intensity level), the treatment switch may be turned off so that the main controller 710 stops generating the microcurrent. As another example, when the power button/treatment switch is pressed and held for a predetermined period of time (e.g., about 2 or 3 seconds) while the skin toning device 70 is operating at a maximum intensity level, the treatment switch may be turned off so that the main controller 710 stops generating the microcurrent. As another example, when the power button/treatment switch 210 is pressed and held for another different predetermined period of time (e.g., 4 or 5 seconds) while the skin toning device 70 is operating, the entire power of the skin toning device 70 may be turned off. Thereafter, the process flow diagram 800 may end. When it is determined in state 870 that the treatment switch is not turned off, the main controller 710 may continue to generate microcurrent at the currently designated level. Thereafter, the main controller 710 may repeat states 830-870.

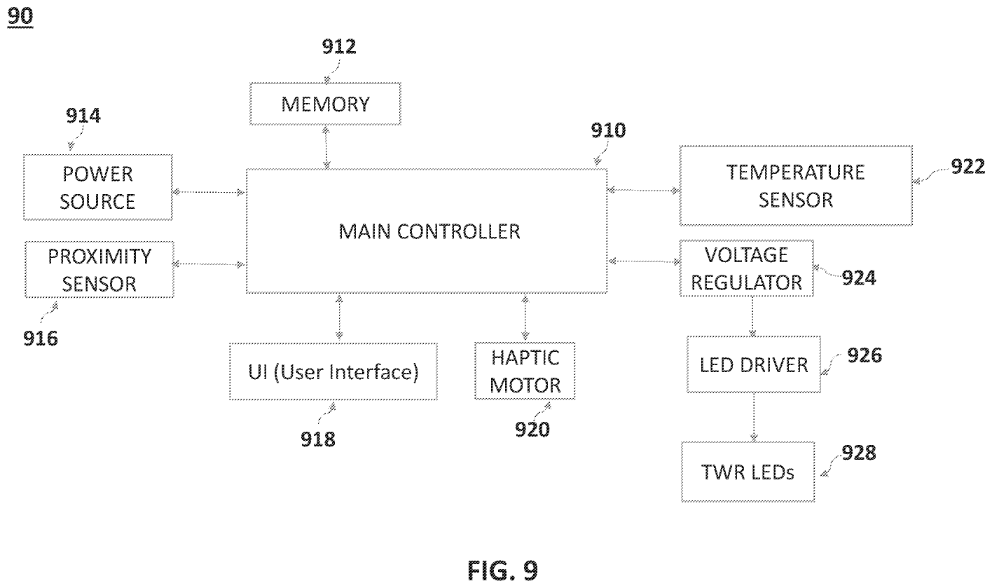

FIG. 9 is an example block diagram of another embodiment of a skin toning device 90. The skin toning device 90 includes a main controller 910, a memory 912, a power source 914, a proximity sensor 916, a user interface (UI) 918, a haptic motor 920, a temperature sensor 922, a voltage regulator 924, an LED driver 926, and LEDs 928 (hereinafter to be interchangeably used with TWR LEDs, where TWR represents the wrinkle reducer.). The skin toning device 90 may be a wrinkle reducer that treats wrinkle, however, the skin toning device 90 may perform other skin treatment. The elements 910-928 may directly or indirectly communicate data with each other. The skin toning device 90 shown in FIG. 9 is merely an example skin toning device, and certain elements may be modified or removed, two or more elements combined into a single element, and/or other elements may be added.

Although the skin toning device 70 of FIG. 7 is a microcurrent based skin treatment device, and the skin toning device 90 of FIG. 9 is a light based skin treatment device, one or more elements of the skin toning device 70 of FIG. 7 may be combined with the skin toning device 90 of FIG. 9. Furthermore, one or more elements of the skin toning device 90 of FIG. 9 may be combined with the skin toning device 70 of FIG. 7. In these embodiments, either of the skin toning devices may use both microcurrent and light for treatment.

Referring to FIG. 9, the main controller 910 may control an overall operation of the skin toning device 90. The main controller 910 can be configured to provide the functionality of the methods described herein, and additional functionality. The main controller 910 may execute instructions stored in the memory 912 to perform various operations of the skin toning device 90 described herein. The main controller 910 may be located in the body, the head, or both in the body and the head of the skin toning device 90.

The memory 912 may communicate data with the main controller 910. The memory 912 may store the executable instructions relating to the various operations of the skin toning device 90 so that the main controller 910 can execute the instructions to perform the various operations of the skin toning device 90.

The power source 914 may provide power to one or more of the remaining elements of the skin toning device 90. The power source 914 may be substantially the same as the power source 714 of the skin toning device 70 shown in FIG. 7. The UI 918 and the haptic motor 920 may be substantially the same as the UI 716 and the haptic motor 718 of the skin toning device 70 shown in FIG. 7.

The proximity sensor 916 may detect proximity of the patient's skin. For example, the proximity sensor 916 may sense proximity when the treatment surface of the skin toning device 90 is near the patient's skin. The proximity sensor 916 may also sense non-proximity when the treatment surface of the skin toning device 90 is away from the patient's skin. When the proximity sensor 916 detects proximity of the patient's skin, the main controller 910 may control the LED driver 926 to enable the TWR LEDs 928 to start light treatment. When the proximity sensor 916 detects run-proximity of the patient's skin, the main controller 910 may control the LED driver 926 to disable the TWR LEDs 928 to stop light treatment. The proximity sensor 916 may be calibrated to provide different levels of sensitivity. For example, assuming that the proximity sensor 916 detects proximity when the skin toning device 90 or the skin treatment surface of the device 90 is within a first distance from a patient's skin, the proximity sensor 916 may be calibrated to detect proximity when the skin toning device 90 or the skin treatment surface of the device 90 is within a second distance from the patient's skin, which is different from the first distance (e.g., either closer or farther). In response to the detection by the proximity sensor 916, the main controller 910 may also be calibrated to enable a more or less number of the TWR LEDs 928, or higher or lower intensity (brightness) of the light for a given number of the TWR LEDs 928. The calibration may be implemented with the firmware update via the smartphone app interface 19 or the software API updates 18 (see FIG. 11) without the need of changing the hardware of the skin toning device 90.

The temperature sensor 922 may detect a temperature of the skin toning device 90. The temperature sensor 922 may be a safety sensor. The temperature sensor 922 may continually monitor the temperature of the skin toning device 90, and may turn off the skin toning device 90 or disable the TWR LEDs 928 when the sensed temperature is greater than a threshold temperature. In some embodiments, the threshold temperature may be, for example, about 45° C. In other embodiments, the threshold temperature may be less than or greater than about 45° C.

The voltage regulator 924 may regulate at least one of a voltage or a current of the LED driver 926 to adjust an intensity or amount of emitted light of the TWR LEDs 928, or enable or disable the TWR LEDs 928. The main controller 910 may control the operation of the voltage regulator 924.

The LED driver 926 may drive the TWR LEDs 928 to adjust an intensity or amount of emitted light of the LEDs 928. The LED driver 926 may also enable or disable the TWR LEDs 928 under the control of the voltage regulator 924 and/or the main controller 910.

The TWR LEDs 928 may be disposed on the treatment surface of the head and provide light emission to the patient's skin for light treatment. The LEDs 928 may be evenly or randomly disposed on the treatment surface of the head. The LEDs 928 may emit various types of light suitable for skin treatment. For example, the TWR LEDs 928 may emit invisible light such as infrared light that can stimulate fibroblasts to increase collagen and elastin, promote circulation, and/or minimize the appearance of pores in the skin. The TWR LEDs 928 may also emit visible light such as red, amber, green, or blue. The red light may stimulate the body's natural healing response, increase collagen and elastin production, boost cell energy, and/or reduce inflammation and damage caused by oxidative stress. The amber light may soothe and calm the skin, and/or improves the look of dull skin. The green light may address skin pigmentation, calm inflammation, and/or help broken capillaries. The blue light may kill some bacteria associated with acne, reduce oil production, and/or calm and soothe skin. Depending on the embodiment, the LEDs 928 may include at least one of an invisible light LED or a visible light LED. Although FIG. 9 shows LEDs, other light source may also be used.

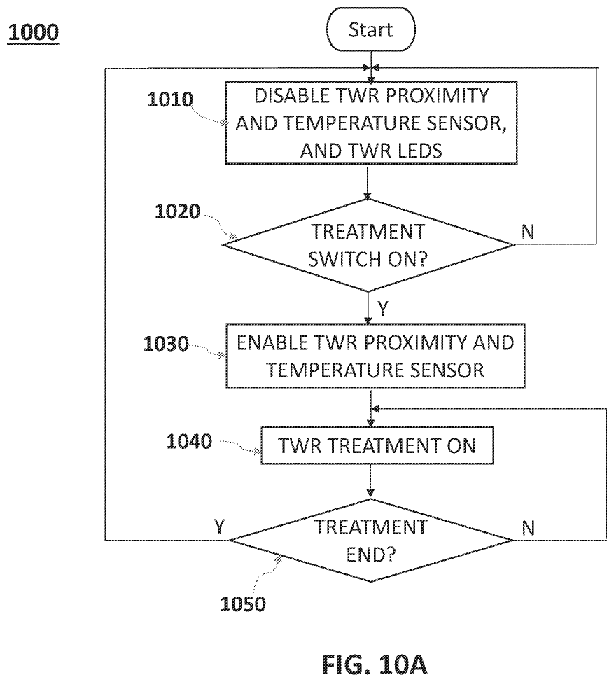

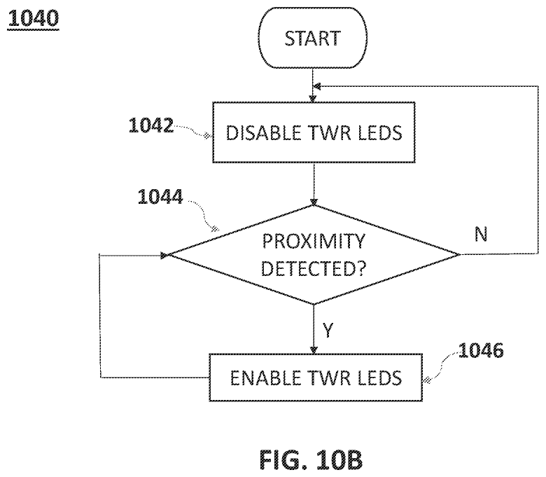

FIGS. 10A and 10B are process flow diagrams 1000 and 1040 of another example method of using the skin toning device 90. The process flow diagrams 1000 and 1040 may be performed by, for example, one or more components (e.g., main controller 910) of the skin toning device 90 of FIG. 9, FIGS. 10A and 10B are merely example process flow diagrams for a method of operating the skin toning device 90, and certain states elements may be removed, other states added, two or more states combined or one state can be separated into multiple states depending on the specification and requirements. For the purpose of convenience, the description will be made based on the main controller 910 shown in FIG. 9 performing the process flow diagram 1000 of FIG. 10A and the process flow diagram 1040 of FIG. 10B.

In state 1010, the main controller 910 may disable the proximity sensor (hereinafter to be interchangeably used with TWR proximity sensor) 916, the temperature sensor (hereinafter to be interchangeably used with TWR temperature sensor) 922, and the TWR LEDs 928 of the skin toning device 90.

In state 1020, the main controller 910 may determine whether the treatment switch is turned on. When it is determined in state 1020 that the treatment switch is turned on, the main controller 910 may enable the TWR proximity sensor 916 and the TWR temperature sensor 922 (state 1030). When it is determined in state 1020 that the treatment switch is not turned on, the main controller 910 may repeat states 1010 and 1020. That is, the main controller 910 may maintain the disabled status of the TWR proximity sensor 916 and the TWR temperature sensor 922 (state 1010), and perform state 1020 again.

In state 1040, the main controller 910 may perform the TWR treatment. State 1040 will be described in more detail by referring to FIG. 10B. In state 1042, the main controller 910 may disable the TWR LEDs 928. In state 1044, the main controller 910 may determine whether proximity is detected. As described above, the TWR proximity sensor 916 may determine proximity based on whether the skin toning device 90 or the skin contacting surface of the skin toning device 90 is adjacent to the patient's skin.

When it is determined in state 1044 that proximity is not detected, the main controller 910 may repeat states 1042 and 1044. That is, the main controller 910 may maintain the disabled status of the TWR LEDs 928 and determine whether proximity is detected again. When it is determined in state 1044 that proximity is detected, the main controller 910 may enable the TWR LEDs 928 and begin TWR treatment (state 1046). While the TWR treatment is being performed, the main controller 910 may continue to determine whether proximity is detected (1044). Depending on whether proximity is detected or not, the main controller 910 may continue to enable the TWR LEDs for continued TWR treatment (state 1046) or disable the TWR LEDs to stop TWR treatment (1042).

Returning back to FIG. 10A, in state 1050, the main controller 910 may determine whether the TWR treatment ends. As described above, the TWR treatment can end when proximity is not detected. The TWR treatment can also end in various other situations. For example, the TWR treatment may end when the treatment turn-off switch is pressed, when a treatment timer is run out, or when the temperature sensor 922 detects a temperature greater than the threshold temperature described above.

FIG. 11 is an example configuration showing different usages of a skin toning device or a skin toning system 110. The skin toning device 110 may include a body or handle 1, a circuit board 10, a charging cradle 5, and an enclosure 23. The skin toning device 110 may also include one or more interchangeable heads 2, 3, 4, and a head housing 20. The skin toning system 110 may also include a smartphone app interface 19, and software API updates 18, both of which can communicate data with the circuit board 10 of the skin toning device 110.

The body 1 may include magnetic attachment mechanism 6, an electrical contact 27, a boost button 24a, one or more indicator LEDs 7, and a power button 24. The one or more indicator LEDs 7 may emit invisible light such as infrared light or various colors of visible light such as red, amber, green, or blue, as described above. The one or more indicator LEDs 7 may emit light having various levels of intensity or brightness. The electrical contact 27 may provide electrical connections between the circuit board 10 of the body 1 and electrical components of the head (2-4, 20). The electrical contact 27 may be configured to protect against water and dust. For example, the electrical contact 27 may be insert molded for improved water and dust ingress protection.

The magnetic attachment mechanism 6 may allow the head (2-4) or head housing 20 to be magnetically attached to and detached from the body 1. In some embodiments, the magnetic attachment mechanism 6 of the body 1 may have N or S polarity whereas the magnetic attachment mechanism 6 of the head (2-4) may have S or N polarity. In other embodiments, one of the body 1 or the head (2-4) may have a magnet and the other one of the body 1 or head (2-4) may have metal that can be attached to and detached from the magnet. Although FIG. 11 shows magnetic attachment mechanism 6, the present disclosure is not limited thereto. For example, the head (2-4) can be coupled to or decoupled from the body 1 via other attachment/detachment mechanism including, but not limited to, screw coupling/decoupling, twist lock or unlock mechanism, push/pull mechanism, etc.

The boost button 24a may be used to perform the boost treatment described above. The power button 24 may be used to turn on or turn off the skin toning device 110. The power button 24 may also be used to change an intensity level of the microcurrent. The intensity level indicators 7 may indicate by gradually turning on the LED indicators 7 depending on, for example, how many times the power button 24 is pressed or how long the power button 24 is pressed and held. Although three LED indicators 7 are shown on FIG. 11, less than or more than three LED indicators can also be provided.

The boost button 24a may have a first tactile characteristic 8 that can provide one or more boost levels by, for example, pressing once or more, or pressing and holding predetermined times, as described above. The power button 24 may have a second tactile characteristic that may be different from the first tactile characteristic so that the patient may recognize which button he or she is pressing without looking at the buttons 24 and 24a, or when the buttons 24 and 24a are indistinguishable or invisible in a dark place. The boost button 24a and the power button 24 may be sealed to prevent water and dust from entering an interior portion of the body 1. The boost button 24a and the power button 24 may be, for example, ultrasonically welded to improve water and dust ingress protection.

The circuit board 10 may include an audio and audio controller 11, a PCB-mounted battery 13, a tactile/haptic motor feedback unit 9, a field-upgradable firmware 21, a Bluetooth module 16, and a wireless charging coil 12. The audio and audio controller 11 may be used to provide audio feedback by providing different types of sound or different volumes of sound. The tactile/haptic motor feedback unit 9 may include a tactile/haptic motor to provide tactile and/or haptic feedback. The tactile/haptic motor feedback unit 9 may include two separate motors respectively providing tactile and haptic feedbacks (or vibration or force feedback) or a single motor that selectively provides tactile or haptic feedback (or vibration or force feedback). The field-upgradable firmware 21 may be upgraded or updated, for example, by downloading updates from the smartphone app 19 thereto. The field-upgradable firmware 21 may also be upgraded or updated, for example, by downloading different versions 22 of the software API updates 18.

The smartphone app 19 may be configured to control various operations of the skin toning device 110 including, but not limited to, turning on and turning off the device 110, changing a treatment intensity level, controlling a boost treatment, proximity sensor calibration, or adjusting feedback mechanism (e.g., audio, visual, haptic). The smartphone app 19 may also be used to log usages of the patient (TWR treatment or microcurrent treatment, particular heads, intensity levels used, frequency of boost treatment, duration of usage, etc.). These usages may be transmitted to a healthcare provider's computer or server for potential consultation with healthcare provides.

The charging cradle 5 may be used as a base or holder for the body 1 of the skin toning device 110 while also functioning as a charging base. For example, when the skin toning device 110 is not used, the device 110 may be put in the charging cradle 5 which can wirelessly charge the device 110. The charging base may include a wireless charging coil 17 that interacts with the charging coil 12 of the circuit board 10 to provide wireless charging (e.g., inductive charging).

The body 1 may also include a magnet holder 25 that houses the magnet 6. The magnet holder 25 may also include a breather port. The breather port may help the internal pressure of the skin toning device 110 maintained substantially similar to or substantially the same as an ambient pressure. This may protect the skin toning device 110 when a patient travels by air while carrying the device 110.

The TWR head 2 may be used to provide TWR treatment by emitting light from LEDs. The TWR head 2 may include a current controller that controls current flow into LEDs so that light emission of the LEDs is adjusted in response to the current flow. The TWR head 2 may also include the proximity sensor 14 that detects proximity described above. The TWR head 2 may include an enclosure 23. The enclosure 23 may be sealed to prevent water and dust from entering an interior portion of the head 2. The enclosure 23 may be, for example, ultrasonically welded to improve water and dust ingress protection.

The head (or FT head) 3 may be used to treat a relatively large skin area, such as forehead or cheek. FT indicates a facial trainer. The head (or ELE head) 4 may be used to treat a relatively small skin area, such as eyelid or lip. ELE indicates an eye and lip enhancer. Although FIG. 11 shows that each of the FT head 3 and the ELE head 4 includes two probes having the same size, the present disclosure is not limited thereto. For example, at least one larger probe, and at least one smaller probe may be installed in the same head. such that the at least one larger probe can be used for treating a larger skin area and the at least one larger probe can be used for treating a smaller skin area, without the need of replacing one head with another head.

Each of the FT head 3 and ELE head 4 may include one or more stimulators or probes 26. The stimulators or probes 26 may include a skin treatment surface at least a portion of which has a surface roughness of about N3 to about N12 as described above. The stimulators or probes 26 may receive microcurrent that flows along the skin treatment surface when the stimulators or probes 26 contact the patient's skin. An outer surface of the stimulators or probes 26 may be bead blasted and have a chrome-plated finish to improve scratch resistance and product longevity. The stimulators or probes 26 may include an enclosure 23. The enclosure 23 may be sealed to prevent water and dust from entering an interior portion of each of the FT head 3 and ELE head 4. The enclosure 23 may be, for example, ultrasonically welded to improve water and dust ingress protection. Each of the FT head 3 and ELE head 4 may be attached to or detached from the body 1 using attachment/detachment mechanism described above.

The head housing 20 may be used to support or accommodate a variety of heads including possible new future heads. In some embodiments, the head housing 20 may include only TWR LEDs or only microcurrent based head (such as FT or ELE head). In other embodiments, the head housing 20 may include both TWR LEDs and microcurrent based head (such as FT or ELE head). The head housing 20 may also accommodate future head functionalities including, but not limited to, a heating head, a cooling head, a vibrating head, a massage head, etc.

FIG. 12 is an example formula of a conductive gel that can be used for a skin toning device according to some embodiments. The conductive gel formula shown in FIG. 12 is merely an example formula, and other formulas are also possible. Furthermore, some materials may be removed, others may be added. For example, one or more of Part A materials, Part B materials, Part C materials, Part D materials, or Part E materials may be replaced with another material. As another example, one or more of Part A materials may be replaced with another material. As another example, one or more of Part B materials may be replaced with another material. As another example, one or more of Part C materials may be replaced with another material. As another example, one or more of Part D materials may be replaced with another material. As another example, one or more of Part E materials may be replaced with another material.

Moreover, weight ratios of the materials may also be changed. In some embodiments, the conductive gel formula may be configured to have a conductivity value of, for example, at least about 1.8 milliSiemens/cm. For example, the conductivity value can be in the range of about 1.8 milliSiemens/cm to about 6.5 milliSiemens/cm. This conductivity value may generally allow for a comfortable microcurrent treatment. However, depending on the embodiment, the conductive value less than about 1.8 milliSiemens/cm or greater than about 6.5 milliSiemens/cm may also be possible.

The conductivity value can be in the range of about 1.8 milliSiemens/cm-about 2.3 milliSiemens/cm, about 2.3 milliSiemens/cm-about 2.8 milliSiemens/cm, about 2.8 milliSiemens/cm-about 3,3 milliSiemens/cm, about 3.3 milliSiemens/cm-about 3.8 milliSiemens/cm, about 3.8 milliSiemens/cm-about 4.3 milliSiemens/cm, about 4.3 milliSiemens/cm-about 4.8 milliSiemens/cm, about 4.8 milliSiemens/cm-about 5.3 milliSiemens/cm, about 5.3 milliSiemens/cm-about 5.8 milliSiemens/cm, about 5.8 milliSiemens/cm-about 6.3 milliSiemens/cm, about 6.3 milliSiemens/cm-about 6.5 milliSiemens/cm, or any other range between the above described ranges, or values. The conductivity value can also be in the range of about 1.8 milliSiemens/cm-about 2.8 milliSiemens/cm, about 2.8 milliSiemens/cm-about 3.8 milliSiemens/cm, about 3.8 milliSiemens/cm-about 4.8 milliSiemens/cm, about 4.8 milliSiemens/cm-about 5.8 milliSiemens/cm, about 5.8 milliSiemens/cm-about 6.5 milliSiemens/cm, or any other range between the above described ranges. The conductivity value can also be in the range of about 1.8 milliSiemens/cm-about 3.8 milliSiemens/cm, about-3.8 milliSiemens/cm-about 5.8 milliSiemens/cm, about 5.8 milliSiemens/cm-about 6.5, or any other range between the above described ranges. The conductivity value can also be in the range of about 1.8 milliSiemens/cm-about 4.15 milliSiemens/cm, about 4.15 milliSiemens/cm-about 6.5 milliSiemens/cm, or any other range between the above described ranges. Each of these conductive values of the conductive gel may be combined one or more of: any one of the microcurrent treatment voltage ranges, any one of the microcurrent treatment current ranges, any one of the microcurrent treatment frequency ranges, any one of the microcurrent pulse ranges, or any one of the microcurrent duty cycle ranges, described above, to provide certain benefits including, but not limited to, improved user comfort and/or skin treatment performance.

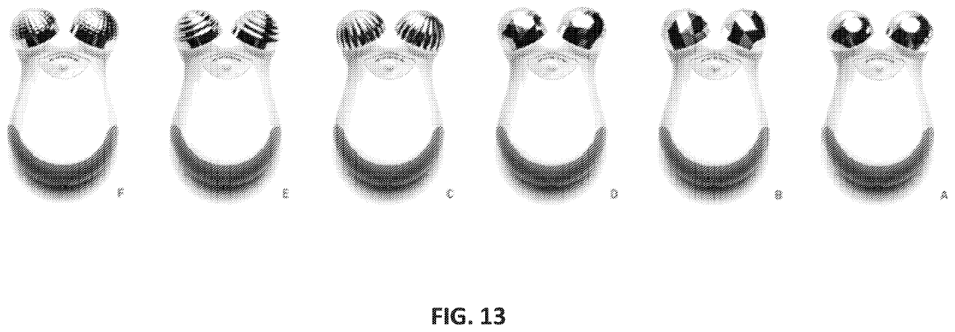

FIGS. 13A-13F show various embodiments of a skin toning device with various stimulator configurations. The stimulator configurations can be implemented within any of the devices described herein, or other skin toning devices. FIG. 13F illustrates a stimulator with a probe that includes a plurality of dimples forming a non-smooth surface extending from its apex. The dimples shown are not necessarily to scale, and in some embodiments, can correspond with the roughness ranges and values described herein. The dimples can be formed by various methods including, but not limited to, bead blasting, form casting, molded, stamped, and/or coated to create a textured surface that is below a threshold of roughness that would be unpleasant to a user. As such, the devices can include a dimpled skin treatment surface. The dimples can be similar size with respect to each other, or can be different sizes. For example, the dimples can increase in size from the apex, extending towards the outer circumference of the stimulator as shown. FIG. 13E illustrates a stimulator with a plurality of circumferentially-extending ribs spaced from each other from the apex to the outer circumference of the stimulator. FIG. 13D illustrates a stimulator with a plurality of ribs extending radially from the apex to the outer circumference of the stimulator, with the ribs spaced circumferentially from each other. FIGS. 13B and 13C illustrate stimulators with a multi-faceted outer shell. FIG. 13A illustrates a stimulator with crimps along its outer circumference.

For purposes of this disclosure, certain aspects, advantages, and novel features are described herein. Not necessarily all such advantages may be achieved in accordance with any particular embodiment. Thus, for example, those skilled in the art will recognize that the disclosure may be embodied or carried out in a manner that achieves one advantage or a group of advantages as taught herein without necessarily achieving other advantages as may be taught or suggested herein.

Conditional language, such as “can,” “could,” “might,” or “may,” unless specifically stated otherwise, or otherwise understood within the context as used, is generally intended to convey that certain embodiments include, while other embodiments do not include, certain features, elements, and/or steps. Thus, such conditional language is not generally intended to imply that features, elements, and/or steps are in any way required for one or more embodiments. Nor is such conditional language generally intended to imply that one or more embodiments necessarily include logic for deciding, with or without user input or prompting, whether these features, elements, and/or steps are included or are to be performed in any particular embodiment. In addition, reference to “one embodiment,” “another embodiment,” etc. is not generally intended to imply that embodiments described herein are separate and distinct, and/or mutually exclusive of one another. Thus, embodiments described herein may contain common elements, features and/or steps.

Conjunctive language such as the phrase “at least one of X, Y, and Z,” unless specifically stated otherwise, is otherwise understood with the context as used in general to convey that an item, term, etc. may be either X, Y, or Z. Thus, such conjunctive language is not generally intended to imply that certain embodiments require the presence of at least one of X, at least one of Y, and at least one of Z.

Language of degree used herein, such as the terms “approximately,” “about,” “generally,” and “substantially” as used herein represent a value, amount, or characteristic close to the stated value, amount, or characteristic that still performs a desired function or achieves a desired result. For example, the terms “approximately”, “about”, “generally,” and “substantially” may refer to an amount that is within less than 10% of, within less than 5% of, within less than 1% of, within less than 0.1% of, and within less than 0.01% of the stated amount. As another example, in certain embodiments, the terms “generally parallel” and “substantially parallel” refer to a value, amount, or characteristic that departs from exactly parallel by less than or equal to 15 degrees, 10 degrees, 5 degrees, 3 degrees, 1 degree, or 0.1 degree.

The scope of the present disclosure is not intended to be limited by the specific disclosures of preferred embodiments in this section or elsewhere in this specification, and may be defined by claims as presented in this section or elsewhere in this specification or as presented in the future. The language of the claims is to be interpreted broadly based on the language employed in the claims and not limited to the examples described in the present specification or during the prosecution of the application, which examples are to be construed as non-exclusive. It should be emphasized that many variations and modifications may be made to the above-described embodiments, the elements of which are to be understood as being among other acceptable examples. All such modifications and variations are intended to be included herein within the scope of this disclosure and protected by the following claims.

As used herein, a phrase referring to “at least one of” a list of items refers to any combination of those items, including single members. As an example, “at least one of: a, b, or c” is intended to cover: a, b, c, a-b, a-c, b-c, and a-b-c.

The various illustrative logics, logical blocks, modules, circuits and algorithm steps described in connection with the implementations disclosed herein may be implemented as electronic hardware, computer software, or combinations of both. The interchangeability of hardware and software has been described generally, in terms of functionality, and illustrated in the various illustrative components, blocks, modules, circuits and steps described above. Whether such functionality is implemented in hardware or software depends upon the particular application and design constraints imposed on the overall system.

The hardware and data processing apparatus used to implement the various illustrative logics, logical blocks, modules and circuits described in connection with the aspects disclosed herein may be implemented or performed with a general purpose single- or multi-chip processor, a digital signal processor (DSP), an application specific integrated circuit (ASIC), a field programmable gate array (FPGA) or other programmable logic device, discrete gate or transistor logic, discrete hardware components, or any combination thereof designed to perform the functions described herein. A general purpose processor may be a microprocessor, or, any conventional processor, controller, microcontroller, or state machine. A processor also may be implemented as a combination of computing devices, such as a combination of a DSP and a microprocessor, a plurality of microprocessors, one or more microprocessors in conjunction with a DSP core, or any other such configuration. In some implementations, particular steps and methods may be performed by circuitry that is specific to a given function.

In one or more aspects, the functions described may be implemented in hardware, digital electronic circuitry, computer software, firmware, including the structures disclosed in this specification and their structural equivalents thereof, or in any combination thereof. Implementations of the subject matter described in this specification also can be implemented as one or more computer programs, i.e., one or more modules of computer program instructions, encoded on a computer storage media for execution by, or to control the operation of, data processing apparatus.

Various modifications to the implementations described in this disclosure may be readily apparent to those skilled in the art, and the generic principles defined herein may be applied to other implementations without departing from the spirit or scope of this disclosure. Thus, the claims are not intended to be limited to the implementations shown herein, but are to be accorded the widest scope consistent with this disclosure, the principles and the novel features disclosed herein. Additionally, a person having ordinary skill in the art will readily appreciate, the terms “upper” and “lower” are sometimes used for ease of describing the figures, and indicate relative positions corresponding to the orientation of the figure on a properly oriented page, and may not reflect the proper orientation of, e.g., an IMOD display element as implemented.

Certain features that are described in this specification in the context of separate implementations also can be implemented in combination in a single implementation. Conversely, various features that are described in the context of a single implementation also can be implemented in multiple implementations separately or in any suitable subcombination. Moreover, although features may be described above as acting in certain combinations and even initially claimed as such, one or more features from a claimed combination can in some cases be excised from the combination, and the claimed combination may be directed to a subcombination or variation of a subcombination.

Similarly, while operations are depicted in the drawings in a particular order, a person having ordinary skill in the art will readily recognize that such operations need not be performed in the particular order shown or in sequential order, or that all illustrated operations be performed, to achieve desirable results. Further, the drawings may schematically depict one more example processes in the form of a flow diagram. However, other operations that are not depicted can be incorporated in the example processes that are schematically illustrated. For example, one or more additional operations can be performed before, after, simultaneously, or between any of the illustrated operations. In certain circumstances, multitasking and parallel processing may be advantageous. Moreover, the separation of various system components in the implementations described above should not be understood as requiring such separation in all implementations, and it should be understood that the described program components and systems can generally be integrated together in a single software product or packaged into multiple software products. Additionally, other implementations are within the scope of the following claims. In some cases, the actions recited in the claims can be performed in a different order and still achieve desirable results.

The foregoing description and claims may refer to elements or features as being “connected” or “coupled” together. As used herein, unless expressly stated otherwise, “connected” means that one element/feature is directly or indirectly connected to another element/feature, and not necessarily mechanically. Likewise, unless expressly stated otherwise, “coupled” means that one element/feature is directly or indirectly coupled to another element/feature, and not necessarily mechanically. Thus, although the various schematics shown in the Figures depict example arrangements of elements and components, additional intervening elements, devices, features, or components may be present in an actual embodiment (assuming that the functionality of the depicted circuits is not adversely affected).

As used herein, the term “determining” encompasses a wide variety of actions. For example, “determining” may include calculating, computing, processing, deriving, investigating, looking up (e.g., looking up in a table, a database or another data structure), ascertaining and the like. Also, “determining” may include receiving (e.g., receiving information), accessing (e.g., accessing data in a memory) and the like. Also, “determining” may include resolving, selecting, choosing, establishing and the like.

The various operations of methods described above may be performed by any suitable means capable of performing the operations, such as various hardware anchor software component(s), circuits, and/or module(s). Generally, any operations illustrated in the Figures may be performed by corresponding functional means capable of performing the operations.

The various illustrative logical blocks, modules, and circuits described in connection with the present disclosure may be implemented or performed with a general purpose processor, a digital signal processor (DSP), an application specific integrated circuit (ASIC), a field programmable gate array signal (FPGA) or other programmable logic device (PLD), discrete gate or transistor logic, discrete hardware components or any combination thereof designed to perform the functions described herein. A general-purpose processor may be a microprocessor, but in the alternative, the processor may be any commercially available processor, controller, microcontroller or state machine. A processor may also be implemented as a combination of computing devices, e.g., a combination of a DSP and a microprocessor, a plurality of microprocessors, one or more microprocessors in conjunction with a DSP core, or any other such configuration.

The methods disclosed herein comprise one or more steps or actions for achieving the described method. The method steps and/or actions may be interchanged with one another without departing from the scope of the claims. In other words, unless a specific order of steps or actions is specified, the order and/or use of specific steps and/or actions may be modified without departing from the scope of the claims.

It is to be understood that the implementations are not limited to the precise configuration and components illustrated above. Various modifications, changes and variations may be made in the arrangement, operation and details of the methods and apparatus described above without departing from the scope of the implementations.

Although this invention has been described in terms of certain embodiments, other embodiments that are apparent to those of ordinary skill in the art, including embodiments that do not provide all of the features and advantages set forth herein, are also within the scope of this invention. Moreover, the various embodiments described above can be combined to provide further embodiments. In addition, certain features shown in the context of one embodiment can be incorporated into other embodiments as well.

1. A skin treatment device comprising:
a body;
a head connected to the body, the head comprising at least one probe with an outward skin treatment surface configured to contact and treat a skin of a user; and
a controller configured to generate a microcurrent, the controller further configured to control the at least one probe to conduct the microcurrent along the skin treatment surface and to cause the microcurrent to flow along the skin of the user when the at least one probe contacts the skin of the user,
wherein at least a portion of the skin treatment surface comprises a surface roughness of at least about N3.

2. The device of claim 1 wherein the at least one probe comprises at least a partially spherical distal end.

3. The device of claim 1, wherein the at least one probe further comprises an elongated shaft extending from a base of the head to the at least partially spherical distal end.

4. The device of claim 2, wherein the at least partially spherical distal end comprises an approximately semi-spherical distal end.

5. The device of claim 1, wherein the at least one probe comprises two or more probes.

6. The device of claim 5, wherein the two or more probes comprise only two probes.

7. The device of claim 6, wherein the two or more probes are adjacent to each other and connected with an uninterrupted, curvilinear section of the body.

8. The device of claim 5, wherein all of the two or more probes extend from a common distal end of the head.

9. The device of claim 5, wherein at least one of the two or more probes is different in size from another one of the two or more probes.

10. The device of claim 1, where the head is a detachable head magnetically attached to the body.

11. The device of claim 1, wherein the surface roughness is in the range of about N3 to about N12.

12. The device of claim 11, wherein the controller is configured to generate the microcurrent to have one or more characteristics of a voltage in the range of about 0V to about 20V, a current in the range of about 0 μA to about 1000 μA, or a frequency in the range of about 0.15 Hz to about 450 Hz.

13. The device of claim 13, wherein the frequency is in the range of about 8.3 Hz-about 8.4 Hz.

14. The device of claim 1, wherein at least the portion of the skin treatment surface is the entirety of the skin treatment surface.

15. The device of claim 1, wherein the outward skin treatment surface is imperforated.

16. The device of claim 1, wherein the outward skin treatment surface is formed of metal.

17. The device of claim 1, wherein the head comprises a base detachably attached to the body, and wherein the at least one probe is fixed to the base when the skin treatment device is in use.

18. The device of claim 1, further comprising a skin impedance detection sensor configured to detect impedance between the skin treatment surface and the skin of the user when the skin treatment surface contacts the skin of the user.

19. The device of claim 1, wherein the controller is configured to provide feedback to the user when the detected impedance is less than a threshold impedance.

20. The device of claim 19, wherein the feedback comprises an audio feedback, a visual feedback, or a haptic feedback.

21. The device of claim 19, wherein the feedback recommends the user apply a conductive gel or apply an increased amount of the conductive gel to at least one of the skin treatment surface or the skin of the user to lower the impedance between the skin treatment surface and the skin of the user.

22. The device of claim 1, wherein the controller is configured to generate different intensity levels of the microcurrent that are user selectable, and to increase an intensity level from a currently selected one of the intensity levels in response to receiving a boost treatment command.

23-38. (canceled)
